(19) United States
(12) Patent Application Publication
(10) Pub. No.: US 2007/0015234 A1
(43) Pub. Date: Jan. 18, 2007

(54) Whole cell assay for inhibitors of cathepsin k

(76) Inventors: Sylvie Desmarais, Quebec (CA); Jean-Pierre Falgueyret, Riguad (CA); Christophe Mellon, L'lle Bizard (CA)

Correspondence Address: MERCK AND CO., INC, P O BOX 2000, RAHWAY, NJ, 07065-0907, US

(21) Appl. No.: 10/570,925

(22) PCT Filed: Sep. 17, 2004
(86) PCT No.: PCT/CA04/01694

§ 371 Date: Mar. 3, 2006

Related U.S. Application Data
(60) Provisional application No. 60/505,245, filed on Sep. 23, 2003.

(51) Int. Cl. C12Q 1/37; A61K 31/655

(52) U.S. Cl. 435/23; 514/150

(57) ABSTRACT

The present invention relates to a method of identifying and evaluating chemical inhibitors of cathepsin K activity in whole cells. The present invention also relates to probes that are capable of forming irreversible adducts with cathepsin K at the active site. The probes used in the instant invention comprise a radioactive functional group to allow the detection of the irreversible cathepsin K-probe adducts.

### BRIEF DESCRIPTION OF THE INVENTION

The present invention relates to a whole cell assay for identifying and evaluating inhibitors of cathepsin K.

### BACKGROUND OF THE INVENTION

A variety of disorders in humans and other mammals involve or are associated with abnormal bone resorption. Such disorders include, but are not limited to, osteoporosis, glucocorticoid induced osteoporosis, Paget's disease, abnormally increased bone turnover, periodontal disease, tooth loss, bone fractures, rheumatoid arthritis, periprosthetic osteolysis, osteogenesis imperfecta, metastatic bone disease, hypercalcemia of malignancy, and multiple myeloma. One of the most common of these disorders is osteoporosis, which in its most frequent manifestation occurs in postmenopausal women. Osteoporosis is a systemic skeletal disease characterized by a low bone mass and microarchitectural deterioration of bone tissue, with a consequent increase in bone fragility and susceptibility to fracture. Osteoporotic fractures are a major cause of morbidity and mortality in the elderly population. As many as 50% of women and a third of men will experience an osteoporotic fracture. A large segment of the older population already has low bone density and a high risk of fractures. There is a significant need to both prevent and treat osteoporosis and other conditions associated with bone resorption. Because osteoporosis, as well as other disorders associated with bone loss, are generally chronic conditions, it is believed that appropriate therapy will typically require chronic treatment.

Osteoporosis is characterized by progressive loss of bone architecture and mineralization leading to the loss in bone strength and an increased fracture rate. The skeleton is constantly being remodeled by a balance between osteoblasts that lay down new bone and osteoclasts that breakdown, or resorb, bone. In some disease conditions and advancing age, the balance between bone formation and resorption is disrupted; bone is removed at a faster rate. Such a prolonged imbalance of resorption over formation leads to weaker bone structure and a higher risk of fractures.

Bone resorption, is primarily performed by multinuclear giant cells, the osteoclasts. The mechanism by which osteoclasts resorb bone is by an initial cellular attachment to bone tissue followed by the formation of an extracellular compartment or lacunae. The lacunae are maintained at a low pH by a proton-ATP pump. The acidified environment allows for initial demineralization of bone followed by the degradation of bone proteins or collagen by proteases such as cysteine proteases (Delaisse, J. M. et al., 1980, Biochem J 192:365-368; Delaisse, J. et al., 1984, Biochem Biophys Res Commun: 441-447; Delaisse, J. M. et al., 1987, Bone 8:305-313). Collagen constitutes 95% of the organic matrix of bone. Therefore, proteases involved in collagen degradation are an essential component of bone turnover, and the development and progression of osteoporosis.

Cathepsins belong to the papain superfamily of cysteine proteases. These proteases function in the normal physiological as well as pathological degradation of connective tissue. Cathepsins play a major role in intracellular protein degradation and turnover and remodeling. To date, a number of cathepsins have been identified and sequenced from a number of sources. These cathepsins are naturally found in a wide variety of tissues. For example, cathepsins B, P, H, L, K, S, W, and Z have been cloned. Cathepsin K (which is also known by the abbreviation cat K) is also known as cathepsin O and cathepsin O2. See, PCT Application WO 96/13523, Khepri Pharmaceuticals, Inc., published May 9, 1996.

Cysteine protease inhibitors such as E-64 (trans-epoxysuccinyl-L-leucylamide-4-guanidino)butane) are known to be effective in inhibiting bone resorption (Delaisse, J. M. et al., 1987, Bone 8:305-313). Recently, cathepsin K was cloned and found specifically expressed in osteoclasts (Tezuka, K. et al., 1994, J Biol Chem 269:1106-1109; Shi, G. P. et al., 1995, FEBS Lett 357:129-134; Bromie, D. and Okamoto, K., 1995, Biol Chem Hoppe Seyler 376:379-384; Bromme, D. et al., 1996, J Biol Chem 271:2126-2132; Drake, F. H. et al., 1996, J Biol Chem 271:12511-12516). Concurrent to the cloning, the autosomal recessive disorder, pycnodysostosis, characterized by an osteopetrotic phenotype with a decrease in bone resorption, was mapped to mutations present in the cathepsin K gene. To date, all mutations identified in the cathepsin K gene are known to result in inactive protein (Gelb, B. D. et al., 1996, Science 273:1236-1238; Johnson, M. R. et al., 1996, Genome Res 6:1050-1055. Therefore, it appears that cathepsin K is probably involved in osteoclast mediated bone resorption.

Cathepsin K is synthesized as a 37 kDa pre-pro enzyme, which is localized to the lysosomal compartment and where it is presumably autoactivated to the mature 27 kDa enzyme at low pH (McQueney, M. S. et al., 1997, J Biol Chem 272:13955-13960; Littlewood-Evans, A. et al., 1997, Bone 20:81-86). Cathepsin K is most closely related to cathepsin S having 56% sequence identity at the amino acid level. The S₂P₂ substrate specificity of cathepsin K is similar to that of cathepsin S with a preference in the P1 and P2 positions for a positively charged residue such as arginine, and a hydrophobic residue such as phenylalanine or leucine, respectively (Bromme, D. et al., 1996, J Biol Chem 271: 2126-2132; Bossard, M. J. et al., 1996,) J Biol Chem 271:12517-12524). Cathepsin K is active at a broad pH range with significant activity between pH 4-8, thus allowing for good catalytic activity in the resorption lacunae of osteoclasts where the pH is about 4-5.

Human type I collagen, the major collagen in bone is a good substrate for cathepsin K (Kafienah, W., et al., 1998, Biochem J 331:727-732). In vitro experiments using antisense oligonucleotides to cathepsin K, have shown diminished bone resorption in vitro probably due to a reduction in translation of cathepsin K mRNA (Inui, T., et al., 1997, J Biol Chem 272:8109-8112. The crystal structure of cathepsin K has been resolved (McGrath, M. E., et al., 1997, Nat Struct Biol 4:105-109; Zhao, B., et al., 1997, Nat Struct Biol 4: 109-11) and selective peptide based inhibitors of cathepsin K have been developed (Bromme, D., et al., 1996, Biochem J 315:85-89; Thompson, S. K., et al., 1997, Proc Natl Acad Sci USA 94:14249-14254). Analyses of these inhibitors should provide further evidence for the role of cathepsin K in bone resorption and in pathological disorders such as osteoporosis.

### SUMMARY OF THE INVENTION

In one embodiment, the present invention relates to a method for identifying a compound as an inhibitor of cathepsin K activity comprising the steps of:
a) incubating eukaryotic host cells possessing endogenous cathepsin K activity with said compound;
b) adding a susbstrate to said eukaryotic host cells in the presence of said compound;
c) incubating said substrate in the presence of said compound;
d) stopping the reaction;
e) quantifying the amount of said substrate in complex with cathepsin K in said eukaryotic host cells; and
f) identifying said compound as an inhibitor of cathepsin K activity.

Another embodiment of the present invention relates to the use of substrates comprised of synthetic probes, which form irreversible adducts with cathepsin K at the active site via an electrophilic functionality of said probe. The present invention is further directed to synthetic probes labeled with moieties selected from the group consisting of a radioactive functional group and a non-radioactive functional group. The present invention further includes a means of identifying a compound as an inhibitor of cathepsin K activity, whereby the ability of the compound to compete with the synthetic probe for the active site of cathepsin K is determined.

Unless otherwise defined, all technical and scientific terms used herein in their various grammatical forms have the same meaning as commonly understood by one of ordinary skill in the art to which this invention belongs. Although methods and materials similar to those described herein can be used in the practice or testing of the present invention, the preferred methods and materials are described below. In case of conflict, the present application, including definitions, will control. In addition, the materials, methods and examples are illustrative only and are not limiting.

Further features, objects and advantages of the present invention are apparent in the claims, and the detailed description that follows. It should be understood, however, that the detailed description and the specific examples, while often indicating preferred aspects of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows labeling of a rabbit synoviocyte HIG-82 cell line by N-[(1S)-1-[[[1-(2-diazoacetyl)butyl]amino]carbonyl]-3-methylbutyl]-4′-[¹²⁵]-[1,1′-biphenyl]-4-carboxamide. Labeling of rabbit cathepsin K was detected by autoradiography after 2-D gel electrophoresis.

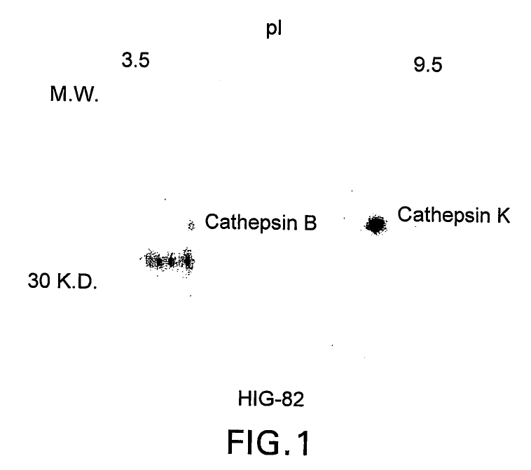

### DETAILED DESCRIPTION OF THE INVENTION

Cell-Based Assay

The present invention is directed to the development of a cell-based assay for identifying compounds as inhibitors of cathepsin K activity.

In a first embodiment of the present invention there is provided a cell-based assay for identifying a compound as an inhibitor of cathepsin K activity which comprises the steps of:
a) incubating eukaryotic host cells possessing endogenous cathepsin K activity with said compound;
b) adding a substrate to said eukaryotic host cells in the presence of said compound;
c) incubating said substrate in the presence of said compound;
d) stopping the reaction;
e) quantifying the amount of said substrate in complex with cathepsin K in said eukaryotic host cells; and
f) identifying said compound as an inhibitor of cathepsin K activity.

In another embodiment of the present invention, the eukaryotic host cells are rabbit synoviocytes (HIG-82 cells).

In another embodiment of the present invention, the incubation of the rabbit synoviocytes with the compound occurs at 37° C. and 5% CO₂.

In another embodiment of the present invention, the substrate comprises a synthetic probe that forms an irreversible adduct with cathepsin K at the active site via an electrophilic functionality on said probe.

In another embodiment of the present invention, the electrophilic functionality of the probe comprises ketones substituted in alpha with a leaving group.

In another embodiment of the present invention, the probe also comprises a functional group to allow the detection of the irreversible cathepsin K-probe adduct.

In another embodiment of the present invention, the functional group comprises a moiety selected from the group consisting of a radioactive functional group and a non-radioactive functional group.

In another embodiment of the present invention, the radioactive functional group is ¹²⁵Iodine, and the non-radioactive functional group is iodine.

In another embodiment of the present invention, the non-radioactive functional group is used to modulate the amount of radioactivity used in the method.

In another embodiment of the present invention, the incubation of the substrate in the presence of the compound occurs at 37° C.+5% CO₂ for a period of about 30 minutes to about 60 minutes.

In another embodiment of the present invention, the reaction is stopped by the addition of a cysteine protease inhibitor.

In another embodiment of the present invention, the cysteine protease inhibitor is E-64D.

In another embodiment of the present invention, the cysteine protease inhibitor is N-[(1S)-1-[[[1-(2-diazoacetyl)butyl]amino]carbonyl]-3-methylbutyl]-4′-[¹²⁵]-[1,1′-biphenyl]-4-carboxamide.

In another embodiment of the present invention, the amount of substrate in complex with cathepsin K is quantified based on measuring the radioactivity acquired by the rabbit synoviocytes.

In another embodiment of the present invention, a compound is identified as an inhibitor of cathepsin K activity based on its ability to compete with the substrate for the active site of cathepsin K in eukaryotic host cells.

The above-described assay method is explicitly directed to testing “a” compound, however it will be clear to a person skilled in the art that such a method can be adapted to testing multiple compounds, e.g., combinatorial libraries to determine if any member of such a collection is inhibitory to cathepsin K activity. Accordingly, the use of collections of compounds, or individual members of such collections is within the scope of this invention.

Definitions

Unless defined otherwise, the scientific and technical terms and nomenclature used herein have the same meaning as commonly understood by a person of ordinary skill to which the invention pertains.

The term “endogenous” describes any naturally-occurring substance that is produced from within an organ or part. In the application herein, the cathepsin K protease is naturally produced by the eukaryotic host cells used in the whole cell assay.

The term “substrate” refers to a compound that is recognized by an enzyme and is a target for its activity. Such a compound can be synthesized, isolated and purified from any chemical or biological source, including recombinant DNA technology. In the application herein the enzyme is a protease and the substrates for detecting its enzymatic activity are comprised of a series of non-naturally occurring chemical compounds that have been designed to form irreversible adducts with cathepsin K at its active site. The substrates used herein on their own cannot be detected. It is therefore necessary to couple the substrates to indicator molecules, thereby enabling identification of the interaction of the substrate with cathepsin K. The coupled substrates and indicator molecules used herein are referred to as collectively as “synthetic probes”. The types of indicator molecules coupled to the synthetic probes include ¹²⁵Iodine. By assaying for the presence or absence of the synthetic probe, one can determine whether a potential inhibitor compound has successfully bound to the active site of cathepsin K, thereby displacing the synthetic probe from the binding site.

The term “adduct” refers to a chemical addition product, or more specifically, to a molecular entity that is formed by the direct combination of two separate molecular entities. The combination occurs in such a way that there is a change in connectivity but no loss of atoms from either of the separate entities that are combined.

The term “electrophilic functionality” refers to an assemblage of atoms that will en compass a partial or full positive charge or dipole than can form an adduct with molecules bearing a partial of full negative charge.

The term “ketones substituted in alpha with leaving group” refers to ketones in which the alpha carbon is substituted with an atom (or array of atoms) whose bond with the alpha carbon of the ketone is such that this atom (or array of atoms) can be displaced by with molecules bearing a partial of full negative charge.

The probes of the present invention can be readily synthesized by techniques known in the art, as well as those methods set forth below, from readily available starting materials.

### EXAMPLE 1

### Synthesis of Methyl N-[(4′-hydroxybiphenyl-4-yl)carbonyl]-L-leucinate

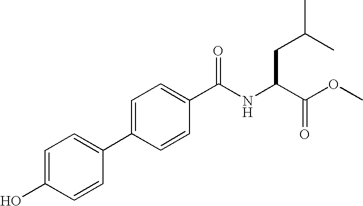 4′-Hydroxybiphenyl-4-carboxylic acid (2.75 g, 12.9 mmoles) and L-leucine methylester hydrochloride (2.8 g, 15.5 mmoles) are suspended in dimethylformamide (20 mL) at room temperature. To the reaction mixture is added HATU (5.2 g, 13.7 mmoles) followed one minute later by triethylamine (7.2 mL, 51.6 mmoles). The reaction is stirred for one hour, diluted with ethyl acetate (200 mL) and 1 N hydrochloric acid (100 mL). The phases are separated and the organic phase washed with 0.1 N hydrochloric acid (100 mL) followed by water (100 mL) then brine (100 mL). The organic phase is dried over magnesium sulfate and concentrated under reduced pressure to afford the title compound in good purity.

### EXAMPLE 2

### Synthesis of Methyl N-[(4′-hydroxybiphenyl-4-yl)carbonyl]-L-leucylnorvalinate

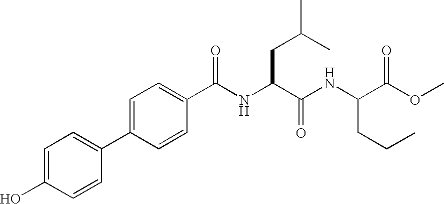 Methyl N-[(4′-hydroxybiphenyl-4-yl)carbonyl]-L-leucinate (2.78 g, 8.1 mmoles) is dissolved in 40 mL of a mixture of tetrahydrofuran, methanol and water to obtain a clear solution. Lithium hydroxyde monohydrate (855 mg, 20.4 mmoles) was added and the reaction mixture was stirred until the disappearance of the starting material by TLC. The reaction was quenched with 1N HCl until pH=1 (50 mL) and the aqueous phase extracted 3 times with dichloromethane (125 mL). The combined organic layers were dried over magnesium sulfate and concentrated under reduced pressure. The crude acid was dissolved in dimethylformamide (25 mL) along with norvalinemethylester hydrochloride (1.64 g, 9.8 mmoles). HATU (3.3 g, 8.6 mmoles) was added followed by triethylamine (4.5 mL, 33 mmoles) one minute later. The reaction was stirred for one hour, diluted with ethyl acetate (200 mL) and 1 N hydrochloric acid (100 mL). The phases are separated and the organic phase washed with 0.1 N hydrochloric acid (100 mL) followed by water (100 mL) then brine (100 mL). The organic phase is dried over magnesium sulfate and concentrated under reduced pressure to afford the title compound in very good purity after a swish in ether.

### EXAMPLE 3

### Synthesis of Methyl N-[(4′-{[(trifluoromethyl)sulfonyl]oxy}biphenyl-4-yl)carbonyl]-L-leucylnorvalinate

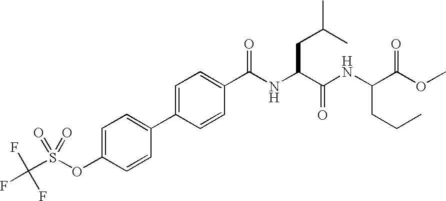 Methyl N-[(4′-hydroxybiphenyl-4-yl)carbonyl]-L-leucylnorvalinate (1.9 g, 4.3 mmoles) was dissolved in dichloromethane (20 mL) and cooled to −20° C. for the addition of triethylamine (1.8 mL, 13 mmoles). Trifluoromethanesulfonic anhydride (0.9 mL, 5.4 mmoles) was added to the reaction mixture over 2 minutes. Examination of the reaction progress by TLC after 10 minutes showed the consumption of all starting material. The reddish reaction mixture was poured onto a mixture of ether (100 mL) and saturated aqueous sodium bicarbonate (75 mL) in a separatory funnel. The phases were separated and the organic phase was successively washed with dilute aqueous sodium bicarbonate (100 mL), 1N hydrochloric acid (100 mL), water (100 mL) and brine (50 ml). The ethereal layer was dried over magnesium sulfate and concentrated under reduced pressure. The residue was purified by flash chromatography over silica gel using 50% Hexanes, 30% ethyl acetate and 20% dichloromethane to obtain the desired material.

### EXAMPLE 4

### Synthesis of Methyl N-{[4′-(trimethylstannyl)biphenyl-4-yl]carbonyl}-L-leucylnorvalinate

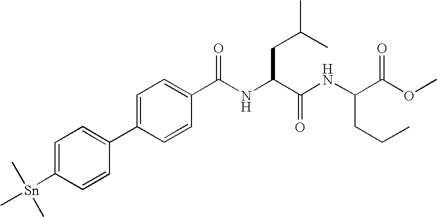 A suspension of Methyl N-[(4′-{[(trifluoromethyl)sulfonyl]oxy}biphenyl-4-yl)carbonyl]-L-leucylnorvalinate (1.9 g, 3.3 mmoles), 2,6-di-tert-butyl-4-methylphenol (few crystals) and lithium chloride (425 mg, 10 mmoles) in dioxane (30 mL) was degassed by three vacuum-Nitrogen flush cycles at room temperature. Hexamethylstannane (1.2 g, 3.7 mmoles) was added followed by palladium tetrakistriphenylphosphine (192 mg, 0.17 mmoles) and the reaction vessel immersed in a 98° C. oil bath for 3 hours. The mixture was poured onto a mixture of ether (100 mL) and saturated aqueous sodium bicarbonate (75 mL) in a separatory funnel. The phases were separated and the organic phase was successively washed with 0.1N hydrochloric acid (100 mL), saturated aqueous sodium bicarbonate (100 mL) and brine (50 mL). The ethereal layer was dried over magnesium sulfate and concentrated under reduced pressure. The residue was purified by flash chromatography over silica gel using a gradient from 70% Hexanes, 30% ethyl acetate to 50% Hexanes, 50% ethyl acetate to obtain the desired material.

### EXAMPLE 5

### Synthesis of N-{[4′-(trimethylstannyl)biphenyl-4-yl]carbony}-L-leucylnorvaline

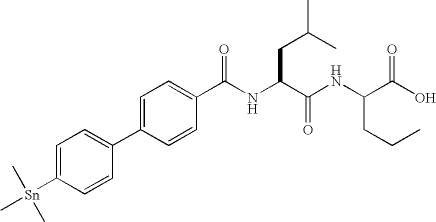 The Methyl N-{[4′-(trimethylstannyl)biphenyl-4-yl]carbonyl}-L-leucylnorvalinate (1.2 g, 2.0 mmoles) is dissolved in 15 mL of a mixture of tetrahydrofuran, methanol and water to obtain a clear solution. Lithium hydroxyde hydrate (130 mg, 3.1 mmoles) was added and the reaction mixture was stirred for nine hours. The reaction was quenched with 1N HCl (20 mL) until pH=1 approximatively and the aqueous phase extracted 3 times with dichloromethane (75 mL). The combined organic layers were dried over magnesium sulfate and concentrated under reduced pressure to give the desired material in fair purity.

### EXAMPLE 6

### Synthesis of N-[(1S)-1-[[[1-(2-diazoacetyl)butyl]amino]carbonyl]-3-methylbutyl]-4′-iodo-[1,1′-biphenyl]-4-carboxamide

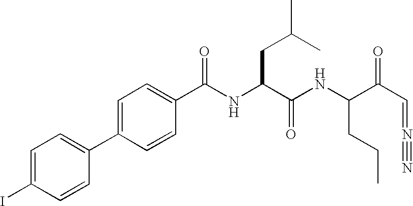 N-{[4′-(trimethylstannyl)biphenyl-4-yl]carbonyl}-L-leucylnorvaline (540 mg, 0.94 mmoles) in dichloromethane (10 mL) at room temperature was treated with an excess of iodine as solution in dichloromethane until the color stayed for 3 minutes. The reaction mixture was diluted with a mixture aqueous sodium bicarbonate (25 mL) and aqueous saturated sodium bisulfite until the system becomes colorless after shaking. The organic phase was dried over magnesium sulfate and concentrated under reduced pressure. The crude acid and N-methylmorpholine (0.25 mL, 2.3 mmoles) in tetrahydrofuran (10 mL) were cooled to 0° C. for the addition of iso-butyl chloroformate (0.14 mL, 1.1 mmoles) and stirred for 20 minutes. An excess of a diethylether solution of diazomethane was added and the reaction stirred at room temperature for 90 minutes and diluted with ether (75 mL) and water (75 mL). The phases are separated and the organic phase was successively washed with dilute aqueous sodium bicarbonate (50 mL), water (50 mL), brine (50 mL) and dried over magnesium sulfate. After concentration under reduced pressure, the residue was purified over silica gel using 50% hexanes, 50% ethyl acetate to afford the cold (not radioactive) desired material.

### EXAMPLE 7

### Synthesis of N-[(1S)-1-[[[1-(2-diazoacetyl)butyl]amino]carbonyl]-3-methylbutyl]-4′-trimethylstannyl-[1,1′-biphenyl]-4-carboxamide

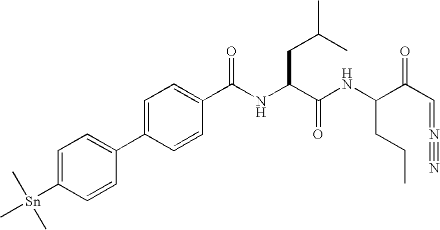 N-{[4′-(trimethylstannyl)biphenyl-4-yl]carbonyl}-L-leucylnorvaline (240 mg, 0.42) and N-methylmorpholine (0.075 mL, 0.6 mmoles) in tetrahydrofuran (4 mL) were cooled to 0° C. for the addition of iso-butyl chloroformate (0.06 mL, 0.5 mmoles) and stirred for 20 minutes. An excess of a diethylether solution of diazomethane was added and the reaction stirred at room temperature for 90 minutes and diluted with ether (50 mL) and water (50 mL). The phases are separated and the organic phase was successively washed with dilute aqueous sodium bicarbonate (30 mL), water (30 mL), brine (30 mL) and dried over magnesium sulfate. After concentration under reduced pressure, the residue was purified over silica gel using 50% hexanes, 50% ethyl acetate to afford the desired material.

### EXAMPLE 8

### Synthesis of N-[(1S)-1-[[[1-(2-diazoacetyl)butyl]amino]carbonyl]-3-methylbutyl]-4′-[¹²⁵I]-[1,1′-biphenyl]-4-carboxamide

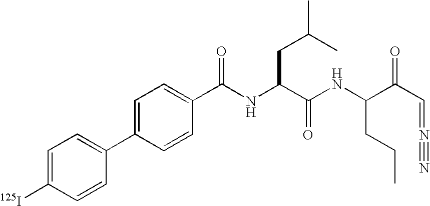 To a room temperature solution of N-[(1S)-1-[[[1-(2-diazoacetyl)butyl]amino]carbonyl]-3-methylbutyl]-4′-trimethylstannyl-[1,1′-biphenyl]-4-carboxamide (50 μL of a 4 mg/mL DMF solution, 0.33 μmol) and 150 μL of DMF was added carrier-free Na¹²⁵I (Draximage, 5 mCi in 0.1 mL of 0.1M NaOH) followed by chloramine-T (20 μL of a 10 mg/mL solution in 1:1 DMF:water, 0.7 μmol). The solution was stirred for 45 min, then quenched with 0.1N NaHSO₃ (40 μL, 4 μmol) and diluted with 150 μL of MeOH. The resulting solution was purified by RP HPLC (Zorbax C18 3.9×150 mm, 1 mL/min, MeOH/water containing 0.01% 2-mercaptoethanol) using the following gradient:

t=0′ 70%

t=5′ 70%

t=9′ 75% (linear gradient)

t=12′ 95% (linear gradient)

t=20′ 95%

t=21′ 70%

The two diastereomers of the title compound elute at 9′ and 10′. The fractions were combined to give 2.9 mCi of the title compound which was stored as a 0.5 μM solution in EtOH+0.01% 2-mercaptoethanol.

### EXAMPLE 9

### Synthesis of 4′-iodobiphenyl-4-carboxylic acid

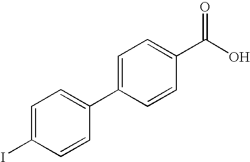 Biphenyl-4-carboxylic acid (13 g, 67 mmoles) in 135 mL of carbon tetrachloride at room temperature is treated with [bis(trifluoroacetoxy)iodo]benzene (32 g, 74 mmoles) followed by finely ground molecular iodine (17 g, 67 mmoles). After 1 h, the reaction formed a gel so 70 mL of carbon tetrachloride were added and stirring resumed for 20 minutes. Solids were filtered and contained only the desired product. The compound was triturated in ether followed by leaving under high vacuum provided the desired product.

### EXAMPLE 10

### Synthesis of Methyl N-[(4′-iodobiphenyl-4-yl)carbonyl]-L-leucinate

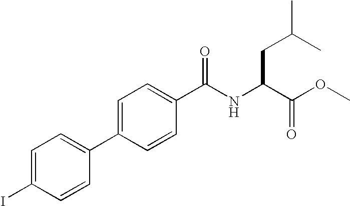 4′-Iodobiphenyl carboxylic acid (15.8 g, 49 mmoles) and L-Leucine methyl ester hydrochloride (10.2 g, 56 mmoles) in dimethylformamide (100 mL) and tetrahydrofuran (100 mL) at room temperature were treated with HATU (19.8 g, 52 mmoles) followed by triethylamine (17 mL, 122 mmoles) one minute later. After 24 h, approximately half of the solvent system was remover under reduced pressure. The mixture is partitioned between half saturated aqueous sodium bicarbonate (300 mL) and ethyl acetate (500 mL). The phases were separated and the organic layer was washed with 1 N hydrochloric acid (250 mL), water (two 250 mL portions) then brine (200 mL). The solution is dried over magnesium sulfate and concentrated under reduced pressure to obtain a solid that is swished in ether with some ethyl acetate.

### EXAMPLE 11

### Synthesis of Methyl N-[(4′-iodobiphenyl-4-yl)carbonyl]-L-leucylnorvalinate

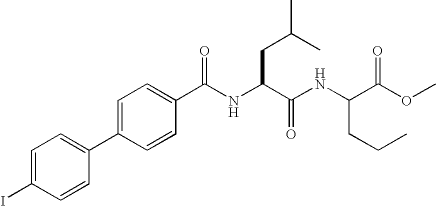 Methyl N-[(4′-iodobiphenyl-4-yl)carbonyl]-L-leucinate (22.5 g, 50 mmoles) in 100 mL of tetrahydrofuran, 40 mL of methanol and 10 mL of water is treated with a suspension of lithium hydroxide monohydrate (3.15 g, 75 mmoles) in 20 mL of boiling water. The reaction was stirred until completion as judged by TLC. Reaction was carefully adjusted to pH 4 approximately by addition of 1 N HCl and most of the solvents were removed under reduced pressure. The concentrated suspension was diluted with dichloromethane (200 mL) and 1 N HCl (150 mL). The phases were separated and the aqueous phase was washed with dichloromethane (150 mL). The combined organic layers were dried over magnesium sulfate and concentrated under reduced pressure. The residue was dissolved in dimethylformamide (100 mL) and treated with racemic norvaline hydrochloride (8.6 g, 51 moles) followed by HATU (19.6 g, 51 mmoles). After one minute, triethylamine (17.5 mL, 125 mmoles) was added and the reaction stirred overnight. The reaction medium was diluted with ethyl acetate (300 mL), diethyl ether (100 mL) and 1N hydrochloric acid (300 mL). The phases were separated and the organic phase was washed with 0.1 N hydrochloric acid (200 mL), two 200-mL portions of water and brine (100 mL). The organic phase was dried over magnesium sulfate and concentrated under reduced pressure to obtain a solid which was purified by trituration in ether.
Rabbit Synoviocytes (HIG-82) Whole Cell Cathepsin K Enzyme Occupancy Assay
Materials
Cell Culture Media
HAM'S F12; 10% FBS; 100 units/mL Pen-Strep
N-[(1S)-1-[[[1-(2-diazoacetyl)butyl]amino]carbonyl]-3-methylbutyl]-4′-[¹²⁵I]-[1,1′-biphenyl]-4-carboxamide (0.5 uM in ethanol containing 0.010% mercaptoethanol)
Sample Buffer (LAEMMLI)

Final buffer concentrations: 75 mM Tris-HCl (pH 6.8); 4.2% glycerol; 1.7% SDS; 3.3% b-mercaptoethanol; bromophenol blue; Add 1 uM E-64 (Sigma # E-3132)

Method

One 75 cm² flask of confluent HIG-82 cells are trypsinized and resuspended in 120 mL of fresh media. Then 2 mL of the cell solution is added per well of a 24 well plate (Nunc). Cells are cultured for 24 hours then media is removed and 500 uL of fresh media is added to each well. Compounds are added 250 fold concentrated in DMSO (2 uL) to obtained the following final concentrations: 10 μM; 1 μM; 0.33 μM; 0.11 μM; 0.037 μM; 0.012 μM; 0.004 μM. Cells and compounds are incubated at 37° C.+5% CO₂ for 1 Hour then N-[(1S)-1-[[[1-(2-diazoacetyl)butyl]amino]carbonyl]-3-methylbutyl]-4′-[¹²⁵I]-[1,1′-biphenyl]-4-carboxamide is added at a final concentration of 1 nM. Stock solution is 0.5 μM in ethanol, the solution is diluted 5 fold in culture media and 5 uL of the solution is added to each well and cells are incubated at 37° C.+5% CO₂ for 30 minutes. Media is removed and 100 μL of sample buffer containing either 1 μM of E64 or cold N-[(1S)-1-[[[1-(2-diazoacetyl)butyl]amino]carbonyl]-3-methylbutyl]-4′-iodo-[1,1′-biphenyl]-4-carboxamide is added to each well. Samples are transferred in eppendorf tubes and stored at −20° C. Samples are heated at 95° C. and loaded on a tris-glycine 10-20% PAGE. Gels are dried for 2 hours and exposed to a KODAK BIOMAX MS film for 2 to 3 hours at −80° C. then the film is scanned with the GS-800 calibrated imaging scanner (BioRad) and signal quantitated with Quantity One software by mean of volume analysis. % Inhibition is calculated relative to the DMSO control and IC50 curve generated with Softmax Pro software (Molecular Devices).

What is claimed is:

1. A method for identifying a compound as an inhibitor of cathepsin K activity comprising the steps of
a) incubating eukaryotic host cells possessing endogenous cathepsin K activity with said compound;
b) adding a substrate to said eukaryotic host cells in the presence of said compound;
c) incubating said substrate in the presence of said compound;
d) stopping the reaction;
e) quantifying the amount of said substrate in complex with cathepsin K in said eukaryotic host cells; and
f) identifying said compound as an inhibitor of cathepsin K activity.

2. The method of claim 1, wherein said eukaryotic host cells are rabbit synoviocytes.

3. The method of claim 2, wherein said incubation of said rabbit synoviocytes with said compound occurs at 37° C. and 5% CO₂.

4. The method of claim 1, wherein said added substrate
comprises a synthetic probe that forms an irreversible adduct with cathepsin K at the active site via an electrophilic functionality on said probe.

5. The method of claim 4, wherein said electrophilic functionality of said probe comprises ketones substituted in alpha with a leaving group.

6. The method of claim 4, wherein said probe comprises a functional group to allow the detection of the irreversible cathepsin K-probe adduct.

7. The method of claim 6, wherein said functional group comprises a moiety selected from the group consisting of a radioactive functional group and a non-radioactive functional group.

8. The method of claim 7, wherein said radioactive functional group is ¹²⁵Iodine.

9. The method of claim 7, wherein said non-radioactive functional group is Iodine.

10. The method of claim 7, wherein said non-radioactive functional group is used to modulate the amount of radioactivity used in said method.

11. The method of claim 1, wherein said incubation of said
substrate in the presence of said compound occurs at 37° C.+5% CO₂ for a period of about 30 minutes to about 60 minutes.

12. The method of claim 1, wherein the reaction is stopped by the
addition of an irreversible inhibitor that acts by binding to the active site cysteine residue of cathepsin S.

13. The method of claim 12, wherein said irreversible inhibitor is E64-D.

14. The method of claim 12, wherein said irreversible inhibitor is N-[(1S)-1-[[[1-(2-diazoacetyl)butyl]amino]carbonyl]-3-methylbutyl]-4′-[¹²⁵I]-[1,1′-biphenyl]-4-carboxamide.

15. The method of claim 1, wherein the amount of said substrate in complex with cathepsin K is quantified based on a method selected from measuring the radioactivity acquired by said eukaryotic host cells.

16. The method of claim 1, wherein said compound is
identified as an inhibitor of cathepsin K activity based on its ability to compete with said substrate for the active site of cathepsin K in said eukaryotic host cells.
